(19) United States
(12) Patent Application Publication
(10) Pub. No.: US 2007/0130796 A1
(43) Pub. Date: Jun. 14, 2007

(54) Foot-stimulation health-promoting device

(75) Inventor: Kazuo Iwata, Aichi (JP)

Correspondence Address: BURR & BROWN, PO BOX 7068, SYRACUSE, NY, 13261-7068, US

(73) Assignee: IWASHO-ORIMONO CO., LTD., NAGOYA-SHI (JP)

(21) Appl. No.: 10/581,762

(22) PCT Filed: May 11, 2005
(86) PCT No.: PCT/JP05/08583

§ 371 Date: Jan. 12, 2007

(30) Foreign Application Priority Data
Mar. 16, 2005 (JP) 2005-075439

(51) Int. Cl. A43B 3/12; A43B 7/26; A61F 5/14

(52) U.S. Cl. 36/11.5; 36/140; 36/94

(57) ABSTRACT

The present invention relates to a foot-stimulation health-promoting device that includes a ring-shaped transverse member 6, which has an expansion and contraction property, and is worn on a portion of the instep of the foot and on the arch of the foot, and a ring-shaped sub-digit engaging member 9 having an expansion and contraction property. These members are mounted on a ring-shaped main body 1 which is equipped with an ankle-engaging portion 2, and a main-digit engaging portion 3 located on at tip, and side lobe portions 4, 5 for connecting them. The transverse member 6 has been sewn on only one of the side lobe portions 4, and the position can be adjusted in a fore-and-aft direction. Both ends of the substrate portion 10 of the sub-digit engaging member 9 have been sewn on the side lobe portion 5.

### TECHNICAL FIELD

The present invention relates to a foot stimulation health promoting device which, when worn on the foot of a human being, can stimulate interdigital areas of the foot, improve blood circulation and palliate muscle fatigue, thereby stabilizing the act of walking and serving to promote health.

### BACKGROUND ART

Conventionally, many health-promoting devices are known which, when worn on the feet of human beings and by stimulating interdigital areas, serve as foot-stimulation health-promoting devices. For example, in Patent Document 1 is described a support made so as to stimulate each of the interdigital areas, by means of making a tip portion of a ring body, which is made of a flexible material having an expansion and contraction capability, branch into a multiple number of branches parallel to the ring body, each of the branching portions being engaged with each of the interdigital areas and the substrate portion of the ring body being engaged with the ankle has been described. However, this invention was not intended as a positive means for stimulating the arch of a foot.

Moreover, in Patent Document 2 is described a support band made so as to stimulate interdigital areas by connecting the tips of a ring-shaped foot internal band, whose substrate portion is engaged with the ankle, and of a ring-shaped foot external band by means of an interdigital area loop, and by means of this interdigital area loop being engaged between the great toe of the foot and the adjoining second toe of the foot. This support band has also been designed mainly for stimulating of the interdigital areas.

Against this background, the present inventor has invented a foot-stimulation heath-promoting device which can stimulate the arch of the foot as well as stimulate interdigital areas, and for which a patent has already been acquired in Japan, as seen in Patent Document 3. This foot-stimulation health-promoting device has found favor with users domestically as well as outside Japan who use it regularly and recognize its merits. Nonetheless, on some occasions problems have been experienced by users. One has been difficulties experienced by non-Japanese wearing shoes because it is necessary to wrap the device around the foot by utilizing a horizontal fastener, a problem when a horizontal fastener used on a foot slips out of position during use, projections of the fastener causing irritation on the foot and making the user feel pain. Other problems have been that the entire device can get twisted after washing and on other occasions it may also be difficult to distinguish whether the correct side of the device is visible, o whether it is inside out.

Patent Document 1: Japanese Published Examined Utility Model Application No. H07-7941 gazette,

Patent Document 2: Japanese Patent Laid-Open No. H10-52472 gazette, and

Patent Document 3: Japanese Patent Publication No. 3464210 gazette.

### DISCLOSURE OF THE INVENTION

### Problem to be Solved by the Invention

Therefore, the object of the present invention is to provide a foot stimulation health-promoting device which can solve the problem of a conventional foot-stimulation health-promoting device described in Patent Document 3, a device that can provide appropriate stimulation to the instep of the foot and to the arch of the foot, that has a shape-retention property, is also easy for an foreigner to wear that can be worn on the foot without the need for a fastener and in which the entire device will not easily get twisted.

### Means for Solving Problem

The foot-stimulation health-promoting device of the present invention made for solving the above-described problems is a foot-stimulation health-promoting device which, when worn on the foot, provides stimulation to interdigital areas of the foot, comprises a ring-shaped main body having an expansion and contraction property, and which is equipped with an ankle-engaging portion, a main-digit engaging portion located on the tip, and side-lobe portions located on both sides for connecting them, a ring-shaped transverse member having an expansion and contraction property and which is fitted to the portions of the instep of the foot and the arch of the foot, and a sub-digit engaging member having an expansion and contraction property, and characterized insofar that the foregoing ring-shaped transverse member has been sewn on one lobe portion of the ring-shaped main body and both the foregoing ends of the substrate portion of the sub-digit engaging member have been sewn on the ring-shaped main body.

It should further be noted that the ring-shaped transverse member can be freely slidably inserted into an insertion portion which has been provided on the other side lobe portion of the ring-shaped main body in a fore-and-aft direction. Moreover, it is preferable that the main-digit engaging portion of the ring-shaped main body be formed so as to be narrower than the ankle-engaging portion and that the side lobe portion on the internal side of the foot be formed so as to be wider than the ankle-engaging portion.

### EFFECTS OF THE INVENTION

The foot-stimulation health-promoting device of the present invention is inserted from the tip of the foot so that the instep of the foot and the arch of the foot correspond to the ring-shaped transverse member, the main-digit engaging portion of the ring-shaped main body is engaged with the interdigital area between the great toe of the foot and the second adjoining toe of the foot and then, the ankle-engaging portion is engaged with the ankle portion, and the ring-shaped sub-digit engaging member having an expansion and contraction property is engaged with the area between the fourth toe and the little toe of the foot, or other interdigital areas, and fitted to the foot.

In this way, when this device is worn on the foot, the main-digit engaging portion provides appropriate stimulation to the area between the great toe and the second toe by an expansion and contraction property of the ring-shaped main body, provides appropriate stimulation between the fourth toe and the little toe of the foot, or other interdigital areas, by an expansion and contraction property possessed by the sub-digit engaging member and can improve blood circulation. Then, when the user walks, the interdigital area between the great toe and the second toe of the foot, and the interdigital area between the fourth toe and the little toe of the foot, or other interdigital areas are stimulated, whereby the gripping force of the toes of the foot is increased and stability in the act of walking can be realized. Furthermore, the ring-shaped transverse member provides a refreshing stimulation to the instep of the foot and to the arch of the foot.

Moreover, since the ring-shaped transverse member has been sewn on one of the side lobe portions, the ring-shaped transverse member can be fitted into a position favored by the user. In this way, the shape retention property of the ring-shaped transverse member is enhanced by regulating the reciprocal relationship between the ring-shaped main body and the ring-shaped transverse member, the entire device is not easily twisted, and wearing of it is facilitated. Particularly, in a case where the ring-shaped transverse member is freely slidably inserted into the insertion portion provided on the other side lobe portion and penetrated in a fore-and-aft direction, the shape-retention property of the entire device is enhanced. Moreover, at a time when it is washed or not in use, even if the right side is turned over, since the insertion portion is on the right side, whether it is the right side or inside out can easily be determined by performing a visual observation of the insertion and penetration portion and wearing it on the foot presents no difficulties.

Furthermore, since both ends of the substrate portion of the sub-digit engaging member have been sewn on the ring-shaped main body and a horizontal fastener is not used in the other portions, it is possible to avoid the conventional problem where the fastener slips out of position at the time when a user walks and the projections of the fastener cause irritation to the foot of the user and thereby trigger pain.

### BRIEF DESCRIPTION OF DRAWINGS

For a more complete understanding of the present invention and the advantages thereof, reference is now made to the following detailed description to be read in conjunction with the accompanying drawings, in which:

FIG. 1 is a perspective view showing an Embodiment of present invention;

FIG. 2 is a side elevation view showing a state where device has been folded;

FIG. 3 is the opposite side of the side elevation view wing a state where the device has been folded;

FIG. 4 is a plan view showing the process of wearing on foot;

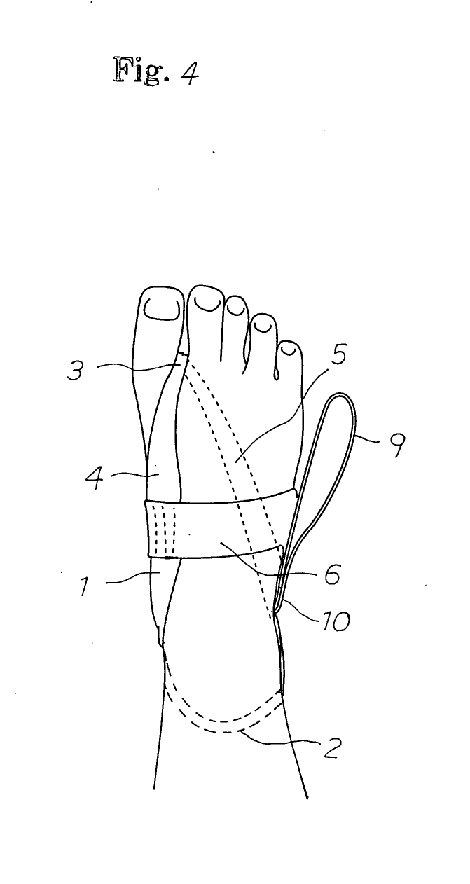

FIG. 5 is a perspective view showing a state where it worn on the foot from the instep side; and

FIG. 6 is a bottom plan view showing a state where it worn on the foot from the outside of the foot.

### DESCRIPTION OF REFERENCE NUMERALS

1 ring-shaped main body,

2 ankle-engaging portion,

3 main-digit-engaging portion,

4 side lobe portion,

5 side lobe portion,

6 ring-shaped transverse member,

7 insertion and penetration portion,

8 thickness portion,

9 sub-digit engaging member, and

10 both ends of the substrate portion.

### BEST MODE FOR CARRYING OUT THE INVENTION

Next, a preferred Embodiment of the present invention will be explained on the basis of drawings. It should be noted that every one of the drawings shows the present invention as used on the right foot. However, it goes without saying that the present invention as used on a left foot is formed in a bilaterally symmetrical manner in relation to the present invention as used on the right foot.

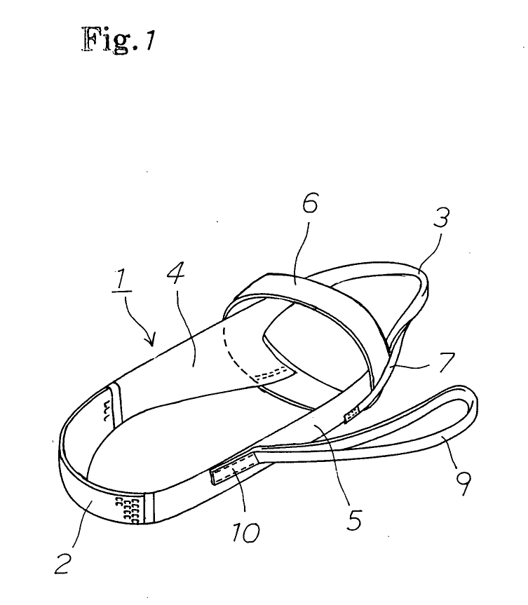

In FIG. 1, the reference numeral 1 denotes a ring-shaped main body made of a material having an expansion and contraction property. As regards the ring-shaped main body 1, a substrate portion side is formed into the ankle-engaging portion 2, and its tip is formed onto the main-digit engaging portion 3, and these two have been connected by means of two of the side lobe portions 4, 5. In this way, the ring-shaped main body 1 has been formed into a ring shape. In this Embodiment, as a material for the ring-shaped main body 1 excluding the ankle-engaging portion 2, a textile having an expansion and contraction property is used. For the surface of the side where it is connected with its foot, a tricot material which has air permeability and a perspiration absorption property, and expands in longitudinal and lateral directions, is used, and for the surface of the outside, a pile material which has air permeability, and the elasticity and is soft to the touch, is used. Furthermore, the edge portion has been folded and subjected to linking finish work, and has been sewn so that it cannot be damaged even if it is repeatedly used. For the ankle-engaging portion 2, a material in the shape of a rubber band is used. By conducting lace stitching of the rubber yarn, the ankle-engaging portion has been made so as to possess elasticity and air permeability. It should be noted that on the internal surface of the ankle-engaging portion 2, a silicon resin for preventing the sliding has been coated.

As shown in FIG. 1 through FIG. 3, the main-digit engaging portion 3 of the ring-shaped main body 1 is formed so as to be narrower than the ankle-engaging portion 2. As a result, the main-digit engaging portion 3 is easily engaged with the area between the great toe of the foot and the second finger of the foot, and appropriate stimulation is easily given to the area between the great toe of the foot and the second toe of the foot. Moreover, only the main-digit engaging portion 3 is formed to a narrow width, and the other portions are made to a comparatively wider width. Thus, the device never cuts into the skin at the time when it is worn on the foot and an excellent sensation can be obtained when the device is worn.

The reference numeral 6 denotes a sing-shaped transverse member having an expansion and contraction property, and this is worn on the portions of the instep of the foot and the arch of the foot. The ring-shaped transverse member 6 is also made of a member in a rubber band, and has a considerable expansion and contraction property, in the same way as the ankle-engaging portion 2 described above. The ring-shaped transverse member 6 has been sewn at a rectangular angle relative to the ring-shaped main body 1 on the side lobe portion 4 of the internal side of the foot of the ring-shaped main body 1. Moreover, in this Embodiment, in the side lobe portion 5 of the outside of the foot, an insertion and penetration portion 7 is provided on which both ends of the band have been sewn, and within a predetermined range the ring-shaped transverse member 6 is freely slidably inserted and penetrated into the insertion and penetration portion 7 in a fore-and aft direction. However, this insertion and penetration portion 7 is not essential, and can also be omitted. In this way, the ring-shaped transverse member 6 has been sewn on one of the side lobe portions 4, whereby the ring-shaped transverse member 6 can be adjusted to a position favored by a user of the present invention. Moreover, by forming the device the constitution described above, the shape-retention property of the ring-shaped instep patch member 5 can be enhanced, and is also becomes easier for a foreigner to wear. Particularly, if the insertion and penetration portion 7 has been provided, even in the case where the top side and the reverse side have been turned inside out at a time when that is being washed or is not in use, the top side and the reverse side can be easily determined by taking advantage of the fact that the insertion and penetration portion 7 is always on the top side.

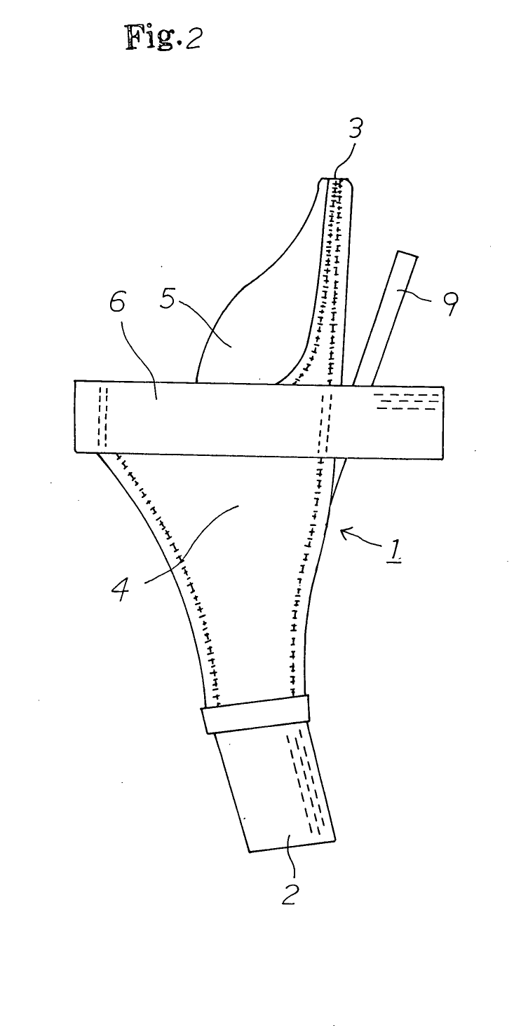

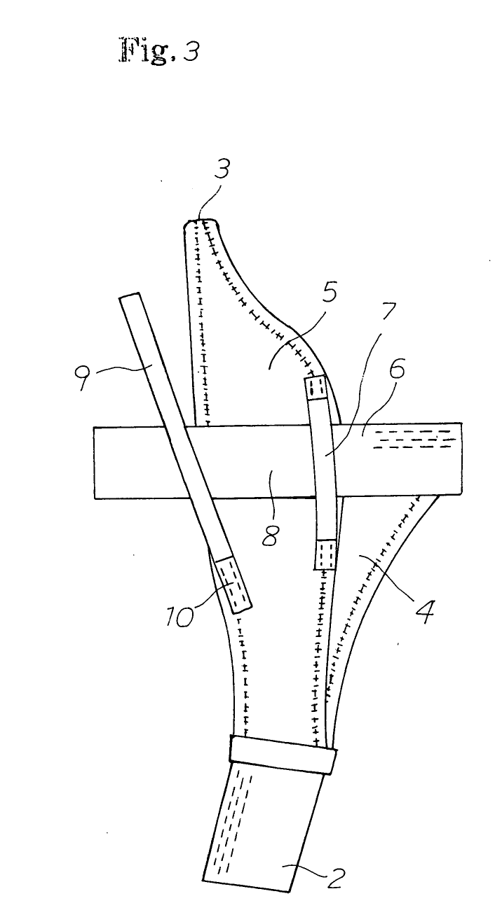

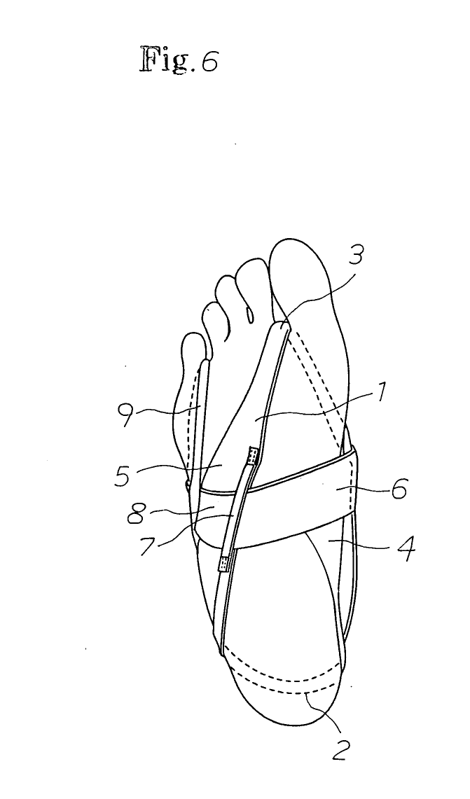

It should be noted that as shown in FIG. 2, the front portion of the side lobe portion 4 of the ring-shaped main body 1 is formed to a width that is greater than the ankle-engaging portion 2. This is to ensure that the side lobe portion supports the portions from the side surface of the foot through the arch of the foot, and for the purpose of enhancing the force of stretching onto legs to the transverse direction of the foot. Moreover, as shown in FIG. 3 and FIG. 6, the tip portion of the side lobe portion 5 on the outside of the foot overlaps with the ring-shaped transverse member 6 and constitutes the thickness portion 8 on the grounding portion of the outside of the arch of the foot, thereby ensuring that a cushion effect is obtained.

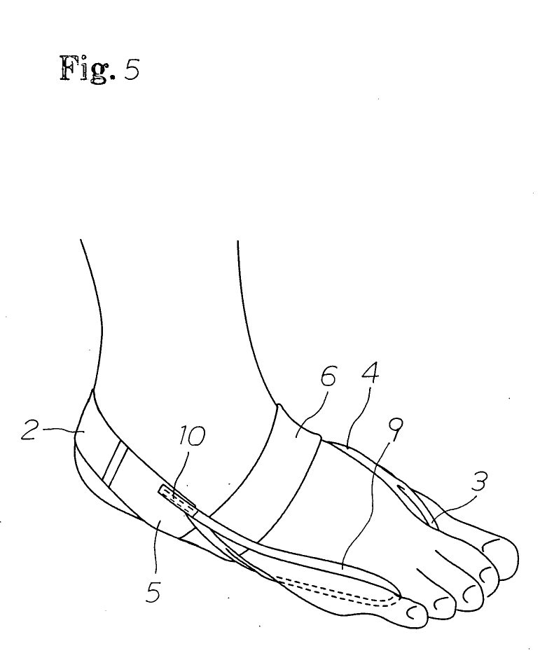

The reference numeral 9 denotes a ring-shaped sub-digit engaging member having an expansion and contraction property. Both edge portions 10 of the substrate portion of the sub-digit engaging member 7 are sewn onto the side lobe portions 5 of the ring-shaped main body 1. As shown in FIG. 5 and FIG. 6 the sub-digit engaging member 7 can become engaged from the instep onto the area between the fourth toe and little toe, and by virtue of the tensile strength of elastic band, can apply an appropriate degree of stimulation to the area between the fourth toe and the little toe. Furthermore, since both ends of the substrate portion of the sub digit engaging member have been sewn on the ring-shaped main body and a horizontal fastener is not used in the other portions, it is possible to avoid the conventional problem whereby the fastener slips out of position at the time when the user walks and the projections of the fastener cause irritation on the foot of the user and cause the user pain.

In a foot-stimulation health-promoting device of the present invention constituted in this way, if it is worn on the foot as shown in FIG. 5 and FIG. 6, an appropriate stimulation to the area between the great toe and the second toe can be given by means of the main-digit engaging portion 3, by virtue of the stretching and contraction property of the ring-shaped main body, and appropriate stimulation is given between the fourth toe and the little toe of the foot, or other interdigital areas, by utilizing the expansion and contraction property possessed by the sub-digit engaging member. Blood circulation can be improved. Moreover, the ring-shaped transverse member 6 gives stimulation to the instep of the foot and the arch of the foot.

Moreover, since the ring-shaped transverse member 6 has been sewn on one of the side lobe portions 4, the ring-shaped transverse member 6 can be adjusted at a position favored by the user the shape-retention property of the entire device is also enhanced, and the device becomes easier to wear. Furthermore, the entire appliance is not easily twisted at a time when it is being washed, or is not in use. Particularly, if the insertion and penetration portion 7 has been previously provided as an Embodiment, even in a case where the top side and the other side are turned inside out, the top side can be easily determined by taking advantage of the fact that the insertion and penetration portion 7 is on the top side. Furthermore, since a fastener is not utilized in the foot-stimulation health-promoting device of the present invention, the conventional problem does not occur whereby, the fastener slips out of position at the time when the user walks and projections of the fastener cause irritation to the foot and cause pain for the user. It should be noted that since both ends of the substrate portion of the sub-digit engaging member 9 have been sewn on the ring-shaped main body 1, strength cannot be adjusted per se, but adjustment becomes possible by means of slightly shifting the position of the entire device. Since the ring-shaped transverse member 6 has been sewn solely on one of the side lobe portions 4, the entire position of the ring-shaped main body 1 can be slightly shifted without changing the position of the ring-shaped transverse member 6.

As hereto described above, it goes without saying that the best mode for carrying out the invention of the present invention has been described. However, the material and shape of the respective portions are not limited to this Embodiment, and a variety of variations can be performed within a range in which there is no deviation from the scope of the invention.

1. A foot-stimulation health-promoting device which, when worn on a foot, providing stimulation to interdigital areas, said foot-stimulation health-promoting device comprising:
a ring-shaped main body equipped with an ankle-engaging portion and, a main-digit engaging portion on the a tip, and side lobe portions located on both sides for connecting them, a ring-shaped transverse member which has an expansion and contraction property and is fitted on portions of an instep of the foot and an arch of the foot, and a ring-shaped sub-digit engaging member having an expansion and contraction property, wherein
said ring-shaped transverse member has been sewn on one of the side lobe portions of the ring-shaped main body, and both ends of a substrate portion of said sub-digit engaging member have been sewn on the ring-shaped main body.

2. The foot-stimulation health-promoting device as claimed in claim 1, wherein the ring-shaped transverse member has been freely slidably inserted and penetrated into an insertion and penetration portion provided on the other of the side lobe portions of the ring-shaped main body in a fore-and-aft direction.

3. The foot-stimulation health-promoting device as claimed in claim 1, wherein the main-digit engaging portion of the ring-shaped main body is formed so as to be narrower the ankle-engaging portion, and the side lobe portion located on the internal side of the foot is formed so as to be a width greater than the ankle-engaging portion.
